(19) United States
(12) Patent Application Publication
(10) Pub. No.: US 2023/0154571 A1
(43) Pub. Date: May 18, 2023

(54) METHOD AND DEVICE FOR SELECTING A SUBASSEMBLY OF MOLECULES FOR USE IN PREDICTING AT LEAST ONE PROPERTY OF A MOLECULAR STRUCTURE

(71) Applicants: ARIANEGROUP SAS, PARIS (FR); CENTRE NATIONAL DE LA RECHERCHE SCIENTIFIQUE, PARIS (FR); UNIVERSITE CLAUDE BERNARD LYON I, VILLEURBANNE (FR)

(72) Inventors: Raphaël TERREUX, LYON (FR); Charlotte ALLIOD, LYON (FR); Roland DENIS, VILLEURBANNE (FR); Guy JACOB, VERT LE PETIT (FR)

(21) Appl. No.: 16/624,700

(22) PCT Filed: Jun. 22, 2018
(86) PCT No.: PCT/FR2018/051529

§ 371 Date: Dec. 19, 2019

(30) Foreign Application Priority Data
Jun. 22, 2017 (FR) 1700668

(51) Int. Cl. G16C 20/30; G16C 20/40; G16C 20/70

(52) U.S. Cl. CPC G16C 20/30; G16C 20/40; G16C 20/70

(57) ABSTRACT

A selection process is iterative and includes an initialization associating with a so-called current molecule a value of a predetermined molecule descriptor associated with the target molecular structure, and during each iteration of the selection process, the process includes evaluating, for each molecule of a database including a plurality of molecules each associated with a value of the descriptor, a so-called overall similarity measure between the value of the descriptor associated with the molecule and the value of the descriptor associated with the current molecule; selecting molecules from the database having an overall similarity measure greater than a predetermined threshold, the selected molecules being added to the reference subset; and updating the value of the descriptor associated with the current molecule from the values of the descriptors associated with at least some of the molecules belonging to the reference subset.

### CROSS-REFERENCE TO RELATED APPLICATIONS

This application is the U.S. National Stage of PCT/FR2018/051529, filed Jun. 22, 2018, which in turn claims priority to French patent application number 1700668 filed Jun. 22, 2017. The content of these applications are incorporated herein by reference in their entireties

### BACKGROUND OF THE INVENTION

The invention relates to the general field of chemical molecules.

It concerns more particularly the prediction of properties of a molecule with a molecular structure.

The invention thus has a privileged but not limited application in predicting the toxicity of compounds, inert or energetic, even high-energy materials, which, in a known way, are capable of releasing energy in a very short time. Due to the energy released, such energetic materials are of interest to both military and civil fields. They are commonly used today in the production of military vehicles, in the manufacture of gases (e.g. propellant) necessary for the propulsion of missiles and space launchers, or are still used in the automotive industry for the manufacture of airbags, etc.

The entry into force in 2007 of the European regulation REACH (Registration, Evaluation Authorisation of Chemicals) requires manufacturers in the European Economic Area who manufacture, import or use chemical substances in quantities exceeding 1 tonne per year to register these substances at European level. The aim is to identify, evaluate and control all chemical substances manufactured, imported or placed on the European market. This regulation is intended to provide the European Union with the legal and technical means to guarantee a high level of protection against the risks associated with chemical substances. It concerns all chemical substances, whether energetic materials or inert products (e.g. additives, stabilisers, plasticizers, glues, etc.).

There is therefore a need for industry, in order to comply with this regulation, to have techniques to identify the toxic effects that a chemical substance can produce on humans or the environment, and more generally to identify its properties, i.e. its biological activity. The focus here is on chemicals with monomolecular structures, so that hereinafter the expressions (mono)molecular chemicals, (mono)molecular structures or molecules are used interchangeably to refer to these substances.

In vitro or in vivo techniques exist, but they are generally long, complex to implement and very expensive in terms of resources, reagents and detection methods.

There are also other techniques known as in silico techniques that rely on computer tools to predict the properties of a chemical substance (e.g. computer models, computerized calculation methods). The most common in silico techniques use so-called quantitative structure-activity relationships (QSARs), which are algorithms (or program equivalents) that establish a quantitative prediction of the biological activity of a monomolecular chemical substance based on its chemical structure. The biological activity of the molecular substance translated by QSARs is based on experimental results and is specific to a given test, typically correlated to the requirements defined by REACH and/or by The Organization for Economic Cooperation and Development (OECD).

To determine the biological activity of a molecular substance by means of a QSAR, in silico techniques use databases (e.g. public databases), specific to the test under consideration, and comprising a plurality of diversified molecules, harmonised in accordance with REACH and/or OECD regulations (e.g. high-energy molecules database). Various strategies can then be considered.

According to a known strategy, a QSAR is applied directly to the entire database. One of the disadvantages of this first strategy is that the database to which QSAR is applied may contain molecules too different from the molecular substance whose biological activity is being predicted, so the resulting prediction may be incorrect.

Other strategies are based on a search for structural similarity between the molecular substance whose biological activity is being predicted and the molecules listed in the database. This similarity research is based on the assumption that all molecules in the database similar to the molecular substance in question have similar properties, including similar biological activity.

To facilitate the search for structural similarity in the database, it is common to represent molecules by structural keys or fingerprints. These keys are descriptors consisting of a plurality of structural characteristic values that characterize molecular structures. One of the best structural keys known to characterize a molecule is the MACCS 166 structural key (for Molecular ACCess System), published by MDL Information Systems. This structural key characterizes each molecule by relying on a table of 166 molecular fragments chosen sufficiently complex to hope to discriminate between different molecules.

Each MACCS 166 structural key is more precisely a vector comprising 166 components or features, having positive or zero values and reflecting the presence or absence of one of the 166 molecular fragments in the molecule under consideration: thus, a zero value reflects the absence of the corresponding fragment in the structure of the molecule, while a positive value indicates the number of times the corresponding fragment is present within the molecule, or simply its presence within the molecule.

In order to compare two molecular structures with each other, a numerical measure of similarity between the two structures can then be calculated using a predetermined metric. A metric conventionally used in combination with the MACCS 166 structural keys is the Tanimoto metric defined by:

$$T ( X , Y ) = \frac{{\sum }_{i} {X}_{i} ∧ {Y}_{i}}{{\sum }_{i} {X}_{i} ∨ {Y}_{i}}$$

where X and Y denote the two structural keys associated respectively with the two molecular structures being compared and where:
Xᵢ∧Yᵢ is equal to 1 if the components Xᵢ and Yᵢ are both positive, and to 0 otherwise; and
Xᵢ∨Yᵢ is equal to 1 if at least one of the components Xᵢ and Yᵢ is not zero, and otherwise to 0.

Note that this metric is applied by simplifying the MACCS 166 structural key of each molecule to obtain a binary vector, with a null component value reflecting the absence of the corresponding molecular fragment, while a component value equal to 1 reflects the presence of this fragment. The Tanimoto metric thus calculated therefore provides the ratio between the number of components of the keys X and Y common to both molecular structures and the total number of components of the keys X and Y expressed (i.e. to which a non-zero value has been assigned in the keys) for these two molecular structures.

The strategies proposed today in the state of the art use this search for structural similarity in two different ways.

According to one strategy, a search for structural similarity is carried out on the database, leading to the identification of a subset of molecules in the database with minimal similarity to the molecular substance whose properties are to be predicted. Then a QSAR is applied to the subset of molecules thus identified. It is therefore clear that, depending on the similarity threshold that is set for selecting the subset of molecules, it is possible to obtain a subset that does not contain enough molecules to apply QSAR in a relevant way, or on the contrary a subset that contains molecules that are too different from the molecular substance whose properties are being predicted. This can lead to an erroneous prediction.

A known strategy to improve the performance of the above strategy is to identify a subset of molecules in the database from another known subset of molecules (e.g. subset of high-energy molecules used by an industrial company), and to select those molecules in the database that have a minimum similarity with each of the molecules in the known subset. A QSAR is then applied to the subset of the database thus identified from the known subset of molecules. Although this strategy has improved performance, prediction errors may still exist.

### SUBJECT MATTER AND SUMMARY OF THE INVENTION

The invention proposes a strategy for predicting the properties of a molecular substance as an alternative to the strategies proposed in the state of the art and making it possible to obtain a prediction of better quality.

More precisely, the invention proposes, according to a first aspect, an iterative process for selecting a subset of so-called reference molecules to be used to predict at least one property of a so-called target molecular structure, the iterative selection process comprising an initialization step associating with a so-called current molecule a value of a predetermined molecule descriptor, associated with the target molecular structure, and during each iteration of the selection process:
a step of evaluating, for each molecule of a database comprising a plurality of molecules each associated with a value of the descriptor, a measure of so-called overall similarity between the value of the descriptor associated with said molecule and the value of the descriptor associated with the current molecule;
a step of selecting molecules from the database with an overall similarity measure greater than a predetermined threshold, the selected molecules being added to the reference subset; and
a step of updating the value of the descriptor associated with the current molecule from the values of the descriptors associated with at least some of the molecules belonging to the reference subset.

Correlatively, the invention is directed at a device for selecting a subset of so-called reference molecules to be used to predict at least one property of a so-called target molecular structure, the selection device comprising an initialization module configured to associate with a so-called current molecule a value of a predetermined molecule descriptor, this selection device being further configured to activate, during a plurality of successive iterations:
an evaluation module configured to evaluate, for each molecule of a database comprising a plurality of molecules each associated with a value of the descriptor, a measure of so-called overall similarity between the value of the descriptor associated with said molecule and the value of the descriptor associated with the current molecule;
a selection module configured to select molecules from the database having an overall similarity measure greater than a predetermined threshold, the selected molecules being added by said selection module to the reference subset; and
an update module configured to update the value of the descriptor associated with the current molecule from the values of the descriptors associated with at least some of the molecules belonging to the reference subset.

The invention also is directed, according to a second aspect, at a process for predicting at least one property of a so-called target molecular substance comprising:
a step of selecting, by means of an iterative selection process according to the invention, a subset of so-called reference molecules in a database comprising a plurality of molecules each associated with a value of a predetermined molecule descriptor;
a step of predicting at least one property of said target molecular substance from the selected subset of reference molecules.

Correlatively, the invention also concerns a prediction device, configured to predict at least one property of a so-called target molecular substance comprising:
a selection device in accordance with the invention, configured to select a subset of so-called reference molecules from a database comprising a plurality of molecules each associated with a value of a predetermined molecule descriptor;
a prediction module, configured to predict at least one property of said target molecular substance from the selected subset of reference molecules.

It should be noted that no limitation is attached to the molecule descriptor considered in the invention to describe each molecule of the database as well as the target molecular substance. This descriptor can be a descriptor comprising a plurality N of features or components, N denoting an integer greater than or equal to 1, in which case the value of the descriptor is defined by the value of each of its N features. These N features can be, for example, structural features that make it possible to characterize each molecule and, if possible, to discriminate between them. For example, the values of the N features of the molecule descriptor may reflect the presence or absence of N molecular fragments considered in the definition of a MACCS 166 structural key.

Alternatively, other descriptors can be considered, such as other known two-dimensional descriptors (or fingerprints) such as MolPrint2D, BCI, or those defined by the companies Tripos and Scitegic. These fingerprints are in the form of bit vectors, each bit encoding the presence (bit equal to 1) or absence (bit equal to 0) of certain predefined structural fragments in the molecule or other features. The invention also applies to other types of descriptors than 2D fingerprints. For example, a descriptor can be considered as a simple variable (i.e. with a single component/feature), the value of which can be a quantitative or qualitative numerical value. The invention also applies to descriptors with more complex shapes, such as vector, raster and even graphic shapes. Such a descriptor is, for example, a connectivity matrix between a plurality of predetermined atoms indicating for each pair of atoms whether or not a bond is present in the molecule under consideration (the descriptor then includes a plurality of features or features given by the components of the matrix).

Nor are there any limitations attached to the technique used to predict the properties of the target molecular substance from the molecules of the reference subset. This may be a quantitative structure-activity relationship (QSAR) as described above, a neural network, a principal component analysis (PCA) or partial least squares (PLS) method, etc.

The invention therefore proposes a novel way of selecting molecules from the initial database used to predict the properties of a molecular substance, and which makes it possible to select a larger subset of molecules similar to the molecular substance and relevant for predicting its properties. This novel way of selecting molecules is based on an iterative process of searching for similarity, first initiated with the target molecular substance whose properties are being predicted. Then, over the iterations, “virtual” molecules are built from the descriptors of the molecules selected in the initial database during the iterations, and a new similarity search is performed from these virtual molecules. The invention thus leads, through this recursive selection and the consideration of similarities with the molecules in the database, to a more complete and careful selection of the molecules in the database to be used to predict the biological properties of the target molecular substance.

It should be noted that the prediction made by the invention is advantageously adaptive. It can easily use regularly updated public databases that list the properties of different molecules with regard to different tests performed on these molecules.

The number of iterations considered to select the subset of reference molecules can be determined by means of a configurable stopping criterion. In this embodiment, the evaluation, selection and update steps are then repeated until a predetermined stopping criterion is verified. Different stopping criteria can be considered, such as:
a predetermined number of iterations performed;
a predetermined number of molecules reached in the reference subset;
the absence of newly selected molecules in the selection step, i.e. molecules that do not already belong to the reference subset before the selection step. In other words, the reference set is no longer enriched over iterations, so there is no need to continue iterating.

The number of iterations and/or molecules of the reference subset can be calibrated empirically.

The choice of one or other of the above criteria (or another criterion) may depend on several parameters, such as the type of target molecular substance considered, a compromise between the number of selected molecules and the quality of the prediction, the method that will be used to predict the properties of the target molecular substance from the properties of the selected molecules, etc.

In a particular embodiment in which the molecule descriptor comprises N features where N denotes an integer greater than 1, the evaluation step comprises, for each molecule of the database, a step of calculating, for each of the N features of the descriptor, a so-called local similarity measure between the value of this feature of the descriptor associated with said molecule and the value of this feature of the descriptor associated with the current molecule, the overall similarity measure evaluated for said molecule being obtained from the local similarity measurements calculated for this molecule.

For example, the calculation step includes for each feature of the descriptor:
a calculation of a distance between the value of the feature of the descriptor associated with said molecule and the value of the feature of the descriptor associated with the current molecule; and
a conversion of the calculated distance to a real number between 0 and 1 by means of a predetermined conversion function, said number being used as a measure of local similarity for said descriptor feature and said molecule.

Such a calculation step advantageously makes it possible to obtain a more accurate measure of similarity than in the state of the art. It can be easily applied to numerical values (e.g. integers) of descriptor features that are positive or zero, not just binary. This results in a more precise and generic assessment of the similarity between two molecular substances than in the state of the art.

Different distances (algebraic) and conversion functions can be considered to implement the invention.

An example of an algebraic distance that can be considered is d(x,y)=x−y where x and y respectively denote the value of the considered feature of the descriptor associated with said molecule and y denotes the value of the considered feature of the descriptor associated with the current molecule.

However, such a distance, although very simple to calculate, does not distinguish between two descriptor feature values equal to 0 and 1, and two descriptor feature values equal to 10 and 11 having the same difference between them as the values 0 and 1, in other words, it does not make it possible to take into account the fact that the two molecules being compared in these two cases have descriptor feature values having different levels.

To take into account such subtleties and to provide a more precise assessment of the similarity between two molecular substances, in a particular embodiment of the invention, the calculated distance, noted d, can be verified:

$$d ( x , y ) = \{ \begin{matrix}0 if x = y \\ - ∞ if x = 0 and y > 0 \\ + ∞ if x > 0 and y = 0 \\ log ( \frac{x}{y} ) else\end{matrix}$$

where x and y respectively denote the value of the feature of the descriptor associated with said molecule and y denotes the value of the feature of the descriptor associated with the current molecule.

Of course, these examples are only given for illustrative purposes.

In addition, a similarity measure is defined as a real number between 0 and 1, taking by convention the value 0 when the two molecules are considered totally different (i.e. not similar), and the value 1 when they are considered totally identical (i.e. similar). Intermediate values can be considered, representing nuances of similarity between these two extremes. To comply with this definition, different conversion functions can be considered.

Thus, in a particular embodiment, the conversion function, noted f, can check:

$$f ( d ) = exp ( \frac{d}{2 {σ}^{2}} )$$

where d denotes the distance to be converted and a a predetermined real number.

In a particular embodiment, in the evaluation step, the overall similarity measure evaluated for said molecule is the ratio between:
the weighted sum of the N local similarity metrics calculated for the N descriptor features for that molecule, and
twice the sum of the weights applied to the local similarity metrics in said weighted sum less said weighted sum.

This definition of the overall similarity measure makes it possible to take into account several states of expression of the same descriptor feature in the molecules being compared: it is not limited to discerning only two binary states of expression (absence or presence of the descriptor feature), unlike in particular the Tanimoto metric described above and considered in the prior art. In addition, this overall similarity measure favourably considers that the common non-expression of the same descriptor (i.e. zero value for this descriptor for the two compared molecules) is a mark of similarity between the two compared molecules.

To update the current molecule during each iteration of the selection process, different strategies can be considered. This current molecule is a kind of representative of the molecules of the reference subset used in the next iteration to complete the reference subset.

Thus, in a first variant, during the update step implemented during an iteration of the selection process, said at least some of the molecules belonging to the reference subset used for the update include the molecules selected during the selection step of this iteration that did not already belong to the reference set before this selection step.

In other words, according to this first variant, only newly selected molecules are taken into account during the current iteration.

However, this first variant may lead to the selection of molecules from the reference set that are slightly too far apart in terms of the similarity of the target molecular structure.

In a second variant, during the update step implemented during an iteration of the selection process, said at least some of the molecules belonging to the reference subset used for the update include the molecules selected during the selection step of this iteration.

According to a third variant, during the update step implemented during an iteration of the selection process, the at least some of the molecules belonging to the reference subset used for the update include all the molecules belonging to the reference subset at the end of the selection step of this iteration.

The inventors found that the second and third variants mentioned above have a fairly similar behaviour and lead to comparable results in terms of prediction. They also give better results than the first variant.

In addition to different strategies for selecting the molecules considered for the update of the current molecule, different strategies can be considered for determining the values of the descriptor features associated with the updated current molecule.

According to a first variant, in the update step, the value associated with the current molecule of each descriptor feature is updated with an arithmetic or weighted average of the values of that descriptor feature associated with the molecules of said at least some of the molecules belonging to the reference subset.

This first variant leads to descriptor feature values that are somehow “artificial”, and do not correspond to values of features present in the at least some of the molecules of the subset used for the update.

To remedy this aspect, according to a second variant, in the update step, the value associated with the current molecule of each descriptor feature is updated with the most frequent value of that descriptor feature among the values of that descriptor feature associated with the molecules of said at least a some of the molecules belonging to the reference subset, or if a plurality of distinct values verify this condition, with the highest value among this plurality of distinct values.

In a particular embodiment, the different steps of the selection process and/or the prediction process are determined by computer program instructions.

Consequently, the invention also concerns a computer program on an information carrier, which may be implemented in a selection device, respectively in a prediction device, or more generally in a computer, which program contains instructions adapted to the implementation of the steps of a selection process, respectively a prediction process, as described above.

This program can use any programming language, and be in the form of source code, object code, or intermediate code between source code and object code, such as in a partially compiled form, or in any other desirable form.

The invention also covers an information or recording medium readable by a computer and containing instructions from a computer program as mentioned above.

The information or recording medium may be any entity or device capable of storing the program. For example, the medium may include a storage medium, such as a ROM, for example a CD-ROM or a microelectronic circuit ROM, or a magnetic recording medium, for example a hard disk.

On the other hand, the information or recording medium may be a transmissible medium such as an electrical or optical signal, which may be conveyed via an electrical or optical cable, by radio or by other means. The program according to the invention can in particular be downloaded on an Internet-type network.

Alternatively, the information or recording medium may be an integrated circuit in which the program is embedded, the circuit being adapted to execute or be used in the execution of the process in question.

It may also be provided, in other embodiments, that the selection process, the prediction process, the selection device and the prediction device according to the invention may have some or all of the above features in combination.

### BRIEF DESCRIPTION OF THE DRAWINGS

Other features and advantages of the present invention will emerge from the description below, with reference to the appended drawings, which illustrate an exemplary, non-limiting embodiment. In the figures:

FIG. 1 schematically represents a prediction device in accordance with the invention, in a particular embodiment;

FIG. 2 represents the hardware architecture of the prediction device of FIG. 1, in a particular embodiment;

FIG. 3 illustrates the different steps of a selection process in accordance with the invention; and

FIG. 4 illustrates the different steps of a prediction process in accordance with the invention.

### DETAILED DESCRIPTION OF THE INVENTION

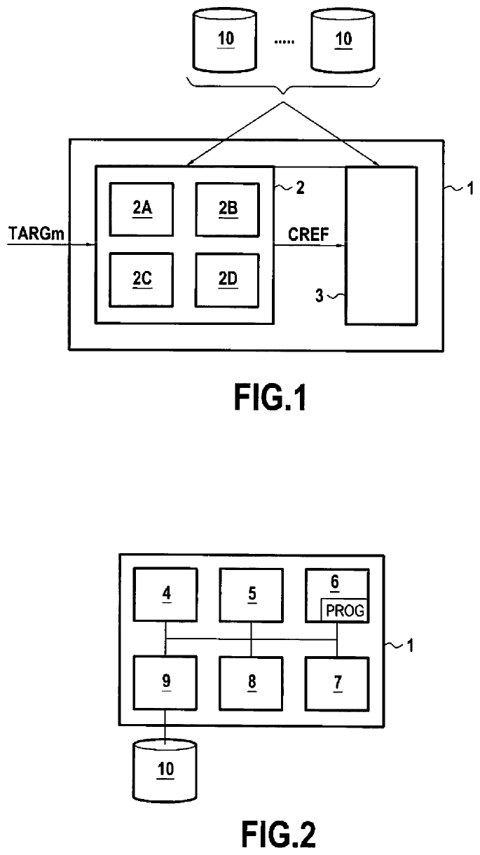

FIG. 1 represents, in its environment, a prediction device 1 in accordance with the invention, in a particular embodiment.

In the example considered in FIG. 1, the prediction device 1 is configured to predict at least one property of a so-called unknown target substance TARGm. It is assumed that this target substance has a monomolecular structure from which it is possible to extract the value of a descriptor comprising a predetermined number N of features (structural here) to characterize the target substance. In the embodiment described here, the descriptor is a vector comprising N=166 features (or components) reflecting the presence or absence in the molecular structure considered of the 166 molecular fragments considered in the definition of the MACCS 166 structural key. In other words, the value of a feature of the descriptor of a molecular substance indicates the presence or absence of the corresponding molecular fragment in the molecular substance.

Alternatively, other descriptors can be considered for the implementation of the invention, as mentioned above (e.g. 2D fingerprints MolPrint2D, BCI, or defined by Tripos and Scitegic, a simple variable whose value can be a quantitative or qualitative numerical value, a connectivity matrix between a plurality of predetermined atoms indicating for each pair of atoms the presence or not of a bond in the molecule under consideration, etc.)

No restriction is attached to the nature of the monomolecular substance in question. For example, this is a high-energy molecule (HEM), but this example is given only as an illustration and the invention applies to all types of molecules.

“Prediction of at least one property of the target substance TARGm” means the prediction of its biological activity. Thus, a property to be predicted can be, for example, a toxicological property of the target substance TARGm, in order to meet the requirements of the European REACH regulation. However, the invention also applies to the prediction of other types of properties of a molecule, such as physicochemical properties (log P or molecular weight), structural properties, absorption, distribution, metabolism and excretion (ADMET) properties, therapeutic properties, etc.

To predict these properties, the prediction device 1 includes:
a selection device 2, in accordance with the invention; and
a prediction module 3.

In the embodiment described here, the prediction device 1 has the hardware architecture of a computer as shown in FIG. 2, and the selection device 2 and the prediction module 3 are software modules installed in a memory of the prediction device 1.

More particularly, the prediction device 1 includes a processor 4, a random-access memory 5, a read-only memory 6, a non-volatile flash memory 7, input/output interfaces 8 (such as a display, keyboard, etc.), as well as communication means 9.

These communication means 9 allow the prediction device 1 to access or download, for example, one or more databases 10, each containing a plurality of molecules. In the embodiment described here, each database 10 considered includes, for each molecule it contains, its name, its molecular structure, the values of the N structural features of the MACCS 166 structural key (in other words, the values associated with the N=166 molecular fragments considered in the MACCS 166 structural key), and the experimental result achieved by this molecule in a given biological test.

Such databases are known per se and are not described in detail here. Each database corresponds to a biological test performed on the molecules it contains. Examples of these databases are described in particular in the document by D. J. Kirkland et al. entitled “Testing strategies in mutagenicity and genetic toxicology: an appraisal of the guidelines of the European Scientific Committee for Cosmetics and Non-Food Products for the evaluation of hair dyes”, Mutat. Res. Toxicol. Environ. Mutagen, vol. 588, pages 88-105, 2005, or in the document by V. Thybaud et al. entitled “Strategy for genotoxicity testing: hazard identification and risk assessment in relation to in vitro testing”, Mutat. Res. Toxicol. Environ. Mutagen, vol. 627, pages 41-58, 2007.

The databases 10 can be hosted on remote servers or stored in a memory of the prediction device 1 (e.g. in its non-volatile memory 7). The communication means 9 of the prediction device 1 allow it to access or download them via a telecommunications network, or to obtain these databases via a recording medium such as a USB (Universal Serial Bus) key or a CD-ROM. For this purpose, they may include a USB port, a network card, a WiFi (Wireless Fidelity) interface, etc.

The read-only memory 6 of the prediction device 1 constitutes a recording medium in accordance with the invention, readable by the processor 4 and on which is recorded here a computer program PROG in accordance with the invention here.

The computer program PROG defines functional modules (and software here), configured to implement the steps of the selection process and the prediction process according to the invention. Alternatively, the two above-mentioned processes can be defined by instructions from two separate programs.

The functional modules defined by the program PROG are based on and/or control the material elements 4-9 of the prediction device 1 mentioned above. They include in particular here, as illustrated in FIG. 1:
an initialization module 2A configured to associate to a so-called current molecule CURm updated during the selection process according to the invention, the value of the MACCS 166 descriptor associated with the target molecule TARGm (the value of the descriptor including here N features);
an evaluation module 2B configured to evaluate so-called “overall” similarity measures between the values of the descriptors associated with a predetermined set of molecules (typically molecules from a database 10) and the value of the descriptor associated with the current molecule CURm;
a selection module 2C configured to select molecules from the considered predetermined set having an overall similarity measure greater than a predetermined threshold, and to add the selected molecules to a so-called reference subset noted CREF; and
an update module 2D configured to update the value of the descriptor associated with the current molecule CURm from the descriptor values associated with at least some of the molecules belonging to the reference subset CREF.

The evaluation 2B, selection 2C and update 2D modules are the modules of the selection device 2, and are configured for the implementation of a selection process according to the invention. They are activated by the selection device 2 repeatedly during a plurality of iterations, and more precisely in the embodiment described here, as long as a predetermined (configurable) criterion is not verified.

The program PROG also defines here the prediction module 3 of the prediction device 1. The prediction module 3 is configured to predict at least one property of the target molecular substance TARGm from the molecules of the reference subset CREF selected by the selection device 2. There are no limitations to the prediction technique implemented by the prediction module 3, such as a QSAR, a neural network, a prediction by principal component analysis, etc. This prediction technique uses the experimental results obtained by the molecules of the reference subset CREF listed in the database 10 from which the subset CREF was extracted.

The different functions of the above-mentioned modules 2A, 2B, 2C, 2D and 3 are now described with reference to the steps of the selection process and the prediction process according to the invention.

As mentioned above, the prediction device 3 predicts at least one property of the molecular substance TARGm from the properties listed in the databases 10 for a plurality of molecules. For the sake of simplicity, we consider here a single database 10 comprising a plurality of molecules and the experimental results obtained by these molecules corresponding to a given biological test.

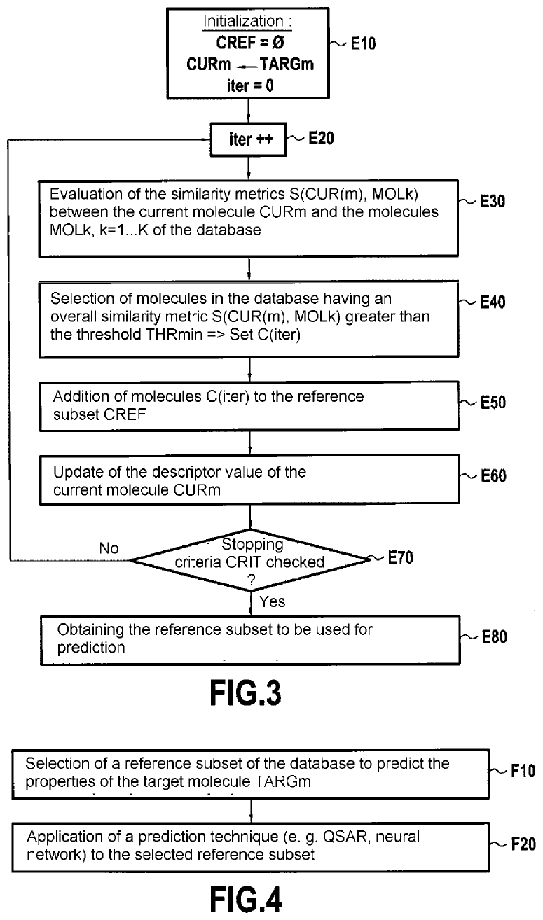

In accordance with the invention, the prediction made by the prediction device 3 is based on a prior selection by the selection device 2 of a reference subset CREF comprising a plurality of molecules extracted from the database 10. FIG. 3 illustrates the main steps of the selection process according to the invention implemented by the selection device 2 to make this selection of the reference subset CREF.

As mentioned above, the selection process is an iterative process, comprising an initialization step (step E10) and implementing a plurality of iterations. In the embodiment described here, iterations follow one another until a predetermined stopping criterion CRIT is verified. The various stopping criteria considered are described in more detail below.

In the initialization step E10 (corresponding to iteration iter=0), the initialization module 2A of the selection device 2 initializes the reference subset CREF to an empty set.

In addition, it initializes the current molecule CURm to the target molecule TARGm whose properties are being predicted. This initialization consists more particularly here in associating with the target molecule TARGm the value of the MACCS 166 structural key associated with the current molecule CURm. This key comprising N=166 features, the initialization consists in other words in associating with the current molecule the values of the N=166 features of the MACCS structural key associated with the target molecule TARGm (i.e. the value of the descriptor consists of the values of its N=166 features). The values of the N MACCS features associated with the current molecule CURm are then designated MACCS(CURm,1), . . . , MACCS(CURm,N).

The selection device 2 then starts the iterations of the selection process (step E20 of index incrementation iter).

More particularly, the selection device 2 evaluates, via its evaluation module 2B, for each molecule MOLk in the database 10 considered, k=1, . . . , K where K is an integer designating the number of molecules listed in the database 10, a so-called overall similarity metric noted S(CURm,MOLk), between the value of the MACCS 166 descriptor associated in the database 10 with this molecule MOLk and the value of the MACCS 166 descriptor associated with the current molecule CURm (step E30). This overall similarity metric is more precisely calculated here between the N values of the N features of the MACCS 166 descriptor associated in the database 10 with the molecule MOLk and the N values of the N features of the MACCS 166 descriptor associated with the current molecule CURm (step E30).

In the embodiment described here, the overall similarity metric S(CURm,MOLk) between each molecule MOLk of the database 10 and the current molecule CURm is evaluated from so-called local similarity measurements ls(CURm,MOLk,n), n=1, . . . N calculated for each of the N features of the MACCS 166 descriptor of the molecules considered.

These local similarity measurements are defined here from a local similarity function Is which, with any pair of integer feature values (x,y), associates a real number ls(x,y) (noted here ls(CURm,MOLk,n) for the nth feature), ranging from 0 to 1 and verifying the following properties:
ls(x,x)=1 for any natural integer x;
ls(x,y)=ls(y,x) for any natural integers x and y.

In the embodiment described here, the function Is results from the composition of a function d assimilable to a geometric distance between the values x and y, and a function f for converting the distance between x and y into a measure of local similarity, i.e.:

ls(x,y)=f(d(x,y))

Different choices are possible for the algebraic distance d(x,y). In the embodiment described here, the evaluation module 2B uses the distance d defined as follows:

$$d ( x , y ) = \{ \begin{matrix}0 if x = y \\ - ∞ if x = 0 and y > 0 \\ + ∞ if x > 0 and y = 0 \\ log ( \frac{x}{y} ) else\end{matrix}$$

In addition, the evaluation module 2B uses as conversion function f, a normalized Gaussian function defined by:

$$f ( d ( x , y ) ) = exp ( \frac{d ( x , y )}{2 {σ}^{2}} )$$

where σ a predetermined real number.

Of course, other distances and conversion functions can be used by the evaluation module 2B to determine local similarity metrics between the N feature values of the considered descriptor of the current molecule CURm and the N feature values of the considered descriptor of the molecule MOLk. However, a conversion function is preferentially chosen that associates to any number of the extended real number line a real value between 0 and 1 such that:
(i) f(+/−∞)=0 (i.e. at an infinite distance between two values of a feature, a value of zero similarity is associated); and
(ii) f(0)=1 (i.e. at a distance of zero between two values of a feature, a unit similarity value is associated).

Thus, during the evaluation step E30, for each molecule MOLk in the database 10, the evaluation module 2 calculates for each feature of the MACCS 166 descriptor indexed by the integer n, n=1, . . . , N, the following local similarity metric:

ls(CURm,MOLk,n)=f(d(MACCS(CURm,n),MACCS(MOLk,n))

where MACCS(CURm,n) and MACCS(MOLk,n) respectively denote the value of the nth feature of the MACCS descriptor of the current molecule CURm and the value of the nth feature of the MACCS descriptor of the molecule MOLk.

Then the evaluation module 2 evaluates the overall similarity metric S(CURm,MOLk) between the molecule MOLk and the current molecule CURm according to the following equation:

$$S ( MOL - A , MOL - B ) = \frac{{\sum }_{n = 1}^{N} ls ( MOL - A , MOL - B , n )}{2 {\sum }_{n = 1}^{N} {w}_{n} - {\sum }_{n = 1}^{N} {w}_{n} ls ( MOL - A , MOL - B , n )}$$

with MOL-A=CURm and MOL-B=MOLk and where wₙ, n=1, . . . , N denote real weights.

It should be noted that this expression of overall similarity results from a search by the inventors for a measure of similarity which, unlike the Tanimoto metric commonly used in the prior art techniques, takes into account different levels of expression of the same descriptor feature (i.e. different values of the same feature) between two compared molecules, and which also considers the common expression of the same descriptor feature (i.e. zero value of that feature) as a mark of similarity between the two compared molecules.

To obtain this expression, the inventors had the judicious idea to use the Jaccard index J(A,B) of two sets A and B defined by:

$$J ( A , B ) = \frac{\vert A∩B \vert}{\vert A ⋃ B \vert} = \frac{\vert A∩B \vert}{\vert A \vert + \vert B \vert - \vert A∩B \vert}$$

where the symbols n and u denote respectively the intersection and the union of the sets A and B, and |X| refers to the cardinal of a set X. They then applied this Jaccard index to two sets A and B consisting of all the pairs formed by each feature index n, n=1, . . . , N and the value of the corresponding feature, associated with two distinct molecules labelled MOL-A and MOL-B (for example here MOL-A=CURm and MOL-B=MOLk). The intersection of the sets A and B can then be written as:

|A∩B|=Σₙ₌₁^{N}wₙ|{n,MACCS(MOL−A,n)}∩{n,MACCS(MOL−B,n)}|

considering that the pairs of molecules MOL-A and MOL-B corresponding to different MACCS descriptor features have empty intersections, and where wₙ, n=1, . . . , N denote real weights. Then by positing:

|{n,MACCS(MOL−A,n)n{n,MACCS(MOL−B,n)}=ls(MOL−A,MOL−B,n)

we get that:

|A⋅B|=Σₙ₌₁^{N}wₙls(MOL−A,MOL−B,n)

Noting that |A|=|B|=N, we obtain from the formula the Jaccard index:

$$J ( A , B ) = \frac{{\sum }_{n = 1}^{N} ls ( MOL - A , MOL - B , n )}{2 {\sum }_{n = 1}^{N} {w}_{n} - {\sum }_{n = 1}^{N} {w}_{n} ls ( MOL - A , MOL - B , n )}$$

By applying this Jaccard index to the molecules CURm and MOLk, the inventors obtained the overall similarity measure used by the evaluation module 2 in the step E30.

It should be noted that a different definition of sets A and B to which the Jaccard index A and B defined above is applied with weights wₙ=1 for n=1, . . . , N, gives the Tanimoto metric.

In the embodiment described here, the evaluation module 2 uses weights wₙ, n=1, . . . , N all equal to 1.

Alternatively, real weights distinct of 1 can be applied by the evaluation module 2. Different strategies can be considered to determine weights wₙ, n=1, . . . , N. For example, these weights can be determined by expertise based on a business knowledge of the relevance of each feature of the descriptor considering the type of target molecule TARGm whose property is to be predicted. These weights can also be determined using statistical methods, in particular classification methods such as linear discriminant analysis (LDA) which makes it possible to determine weights leading to better discrimination between experimentally positive molecules (i.e. which are considered to have responded positively to the toxicity test considered) and experimentally negative molecules (i.e. which are considered to have responded negatively to the toxicity test considered).

Once the overall similarity metrics S(CURm, MOLk) are evaluated for each molecule MOLk in the database 10, the selection device 2, via its selection module 2C, determines which molecules in the database 10 have an overall similarity measure greater than a predetermined threshold THRmin (or equivalently greater than or equal to a predetermined threshold THRmin′) and selects them (step E40).

The molecules thus selected form a set C(iter) of molecules considered similar to the current molecule CURm. The threshold THRmin is a constant parameter here during the iterations of the selection process, and ranges from 0 to 1. It may depend in particular on the type of target molecule TARGm whose properties are being determined (e.g. high-energy molecule, solvent, plasticisers, liquid, etc.). This threshold can be determined beforehand experimentally.

For example, the inventors determined by experimentation that a threshold THRmin=0.85 (or greater than or equal to 0.85) leads to good predictions for different categories of molecules (fillers, plasticizers, liquids, etc.).

Alternatively, the threshold THRmin may change over iterations.

The set of molecules C(iter) selected during the current iteration iter is then added by the selection module 2C to the reference set CREF (step E50). It should be noted that some molecules contained in the set C(iter) may already be present in the reference set CREF, in which case the addition of molecules from the set C(iter) to the reference set CREF is limited to adding only new molecules not already present in the reference set CREF.

Then, in the embodiment described here, the selection device 2, via its update module 2D, updates the value of the MACCS descriptor associated with the current molecule (step E60). This results here in an update of the N values of the features MACCS(CURm,1), . . . , MACCS(CURm,N) of the descriptor associated with the current molecule CURm. In this way, it is a question of defining a new “virtual” molecule that is common for the next iteration, from which a new search for similarity in the database 10 will be carried out.

In accordance with the invention, this update is carried out using the descriptor values of at least some of the molecules present in the reference subset CREF at the end of the step E50.

Different ways of updating the N values of the features MACCS(CURm,n), n=1, . . . , N of the MACCS descriptor can be implemented by the update module 2D. These ways can be distinguished, on the one hand, by the molecules of the reference subset CREF that are used, and, on the other hand, by the way in which the values of the descriptor features of these molecules are combined to obtain the updated values of the current molecule CURm.

In the embodiment described here, the update of the MACCS descriptor feature values of the current molecule CURm is based on the MACCS descriptor feature values of the molecules selected during the current iteration iter, i.e. on the molecules contained in the set C(iter).

In another embodiment, the update of the MACCS descriptor feature values of the current molecule CURm is based on the MACCS descriptor feature values of all molecules belonging to the reference set CREF at the end of the step E50.

In yet another embodiment, the update of the MACCS descriptor feature values of the current molecule CURm is based only on the MACCS descriptor feature values of the molecules newly selected in the selection step E40 implemented in the current iteration iter, i.e. on the MACCS descriptor feature values of molecules belonging to set C(iter) but not already belonging to the reference set CREF before the step E50.

In addition, in the embodiment described here, to update each MACCS descriptor feature value MACCS(CURm,n) of the current molecule CURm, n=1, . . . , N, the update module 2D uses the most frequent value of each feature among the values of this feature associated with the molecules considered for the update. In case of ambiguity, i.e. if several distinct values check this frequency condition, the update module 2D uses the highest value among this plurality of distinct values.

Alternatively, to update each MACCS descriptor feature value MACCS(CURm,n) of the current molecule CURm, n=1, . . . , N, the update module 2D can use an average of the values of this feature associated with the molecules considered for the update (or the integer value closest to this average to obtain integer features), this average being an arithmetic or weighted average.

At the end of this step E60, a new current molecule CURm is obtained on which a new similarity search in the database 10 can be performed during the next iteration.

In the embodiment described here, the selection device 2 checks, at the end of the step E60, whether the stopping criterion CRIT is checked (test step E70). Different stopping criteria can be considered, such as:
a predetermined number ITERMAX of iterations performed;
a number KMAX of molecules reached in the reference set CREF;
the absence of newly selected molecules in the set C(iter) during the selection step E40.

This stopping criterion can be configured. The ITERMAX and KMAX numbers are also configurable and depend in particular on the type of molecules considered.

If the stopping criterion is not verified (answer no to the test step E70), then a new iteration of the selection process is implemented (increment step E20), this iteration including the repetition of the steps E30 to E70 for the new current molecule CURm obtained in the step E60.

If the stopping criterion is verified (answer yes to the test step E70), the iterations of the selection process are interrupted and the reference set CREF is provided to the prediction module 3 for predicting the properties of the target molecular substance TARGm.

It should be noted that if the stopping criterion CRIT considered is a number KMAX of molecules reached in the reference set CREF, the reference set CREF considered is preferentially the one obtained at the end of the iteration so as not to exceed the number KMAX.

FIG. 4 illustrates the different steps of the prediction process implemented by the prediction device 1.

In this figure, the step F10 repeats the steps of the reference subset CREF selection process previously described in reference to FIG. 3 and implemented by the selection device 2 of the prediction device 1.

As mentioned above, the reference set CREF obtained by the selection device 2 is then provided to the prediction module 3. This is configured to predict at least one property of the target molecular substance TARGm from the molecules of the reference set CREF selected by the selection device 2 (step F20).

No limitations are attached to the prediction technique implemented by the prediction module 3 for this purpose. In particular, it may use a QSAR as described above and commonly used in the state of the art, or a neural network, a prediction by principal component analysis, etc. This prediction technique uses the experimental results obtained by the molecules in the reference set CREF and listed in the database 10 from which the set CREF was extracted. The use of such prediction techniques is known per se and is not described in greater detail here.

The prediction device 1 then obtains at the end of the step F20 a prediction of at least one biological property of the target molecular substance TARGm. Other predictions can be made by the prediction device 1 from other databases 10 corresponding to other bioassays.

The invention, via the novel selection process proposed, makes it possible to obtain a reliable prediction of the properties of a molecular substance from the properties of molecules of the same type listed in public databases in particular. The inventors noted an improvement in the predictions obtained compared to state-of-the-art prediction techniques for different categories of molecules (fillers, plasticizers, oxidants, liquids, stabilizers, pyrotechnic components, etc.) and for different regulatory tests known to the skilled person (e.g. AMES mutagenicity test, chromosome aberration test, unscheduled DNA synthesis (UDS) test, carcinogenicity test, etc.). Some results are provided in Annexes 1 to 6 to illustrate the performance of the selection and prediction processes according to the invention.

Annex 1 illustrates prediction results obtained for the AMES test using five different prediction methods. The AMES test is a known mutagenicity test performed on different bacterial cultures to determine whether a molecule has a mutagenic property (indicated in the table in Annex 1 by a symbol “+”, a symbol “−” indicating that the molecule does not have a mutagenic property).

The table presents in its first column data that have been obtained experimentally from the molecules tested. These data have been validated at the level of the European authorities and have been used as a reference to determine the relevance of the predictions made using the different prediction methods tested. For each of these methods, when a result obtained is between 0 and 0.4, it is considered negative, i.e. it reflects the absence of mutagenic property in the molecule tested; when this result is between 0.4 and 0.6, it is considered doubtful; and when this result is greater than 0.6, it is considered negative, i.e. it reflects the presence of the mutagenic property in the molecule tested.

The table in Annex 1 provides the prediction results obtained using the five methods tested for different load molecules: the five prediction methods were each applied to an initial database containing 7723 reference molecules. More precisely:
the table column denoted (1) corresponds to the application of a QSAR to the initial database;
the table column denoted (2) corresponds to the application of a QSAR to a database obtained by selecting from the initial database molecules with a similarity metric (Tanimoto metric) of 0.8;
the table column denoted (3) corresponds to the application of a QSAR to a database obtained by selecting from the initial database molecules with a similarity metric (Tanimoto metric) of 0.8;
the table column denoted (4) corresponds to the application of a QSAR to a database obtained through the iterative selection process according to the invention and applied to the initial database (MACCS 166 structural descriptors). The stopping criteria considered for the iterative process are a maximum of 5 iterations or 600 molecules selected from the initial database. The local and overall metrics described in the detailed embodiment described above were used; and
the table column denoted (6) corresponds to the application of a machine learning algorithm on a database obtained through the iterative selection process according to the invention and applied on the basis of initial data (MACCS 166 structural descriptors). The stopping criteria considered for the iterative process are a maximum of 5 iterations or 600 molecules selected from the initial database. The local and overall metrics described in the embodiment detailed above were used.

It appears from the results obtained for different molecules that the prediction process according to the invention, whether based on a QSAR or a machine learning algorithm, provides very good prediction results (respectively 16 and 17 correct predictions of the 17 performed), and better performance than the other state-of-the-art methods tested (corresponding to columns (2) and (3)).

Annex 2 reflects other prediction results obtained for the AMES test, for different categories of molecules (fillers, plasticizers, oxidants, liquids, stabilizers and pyrotechnic molecules), with the selection and prediction processes according to the invention (“prediction” column of the different tables in Annex 2). The same assumptions as those used in Annex 1 were considered (maximum number of iterations equal to 5, 600 selected molecules maximum, local and overall metrics detailed above, MACCS 166 structural descriptors); the prediction step strictly speaking was performed on the basis of molecules selected using the selection process according to the invention by applying a machine learning algorithm.

Experimental data obtained for the molecules tested are provided for information purposes (“Exp. data” column). The percentages indicated correspond to the reliability of the prediction made thanks to the invention. When this reliability is between 40% and 60%, the result of the prediction is considered doubtful. Above 60%, the prediction is considered correct. Below 40%, the prediction is considered erroneous.

Thus, the various tables produced in Annex 2 show that:
the prediction process led to a correct prediction for all the molecules tested of the load type (i.e. all percentages reported are greater than 60%), for all the molecules tested of the liquid type, and for all the molecules tested of the stabilizer type;
for the sets of molecules tested of pyrotechnic and oxidizing type, only one molecule led to a doubtful prediction (corresponding to a reliability of 58% and 57% respectively).

Annexes 3 to 5 reflect prediction results obtained using the prediction process according to the invention for other known regulatory tests (chromosome aberration test in Annex 3, UDS test in Annex 4, carcinogenicity test in Annex 5). The same assumptions as those used in Annex 2 were considered for the implementation of the processes according to the invention and the interpretation of the results presented.

Annex 6 compares the results obtained using the prediction process according to the invention and another state-of-the-art prediction process known as ACD (Advanced Chemistry Development) Percepta (described in more detail on the web page https://www.acdlabs.com/products/percepta/).

The results concerning the prediction process according to the invention were obtained from two different initial databases (denoted “first test database” and “second test database”). The first test database is the one already used to generate the results reported in Annexes 2 to 5. The first column of results in the table presented in Annex 6 gives the rate of good predictions obtained via the prediction process according to the invention relative to the different molecules tested for the different tests considered. This first column lists the different results illustrated in Annexes 2 to 6 for all categories of molecules considered combined and complements these results for other known regulatory tests (mouse lymphoma assay (MLA), DLT, and reproductive toxicity assay).

Other results obtained on a second initial database are also reported in the table in Annex 6. These results make it possible to compare the performances obtained on the second initial database with the prediction process according to the invention (still according to the same assumptions as described above) with the performances obtained on the same basis with the ACD process. We see that the rate of good predictions obtained with the prediction process according to the invention is about 90% versus 55% for the ACD process.

Annex 1
AMES mutagenicity test
Load molecules

| | | | (2) | | | |
|---|---|---|---|---|---|---|
| | | | Reduced | (3) Reduced | | (5) |
| | | (1) | database | database | (4) | Invention |
| | | Complete | with | with | Invention | process + |
| | Experimental | database + | similarity | similarity | process + | machine |
| Molecule name | data | QSAR | 0.8 + QSAR | 0.85 + QSAR | QSAR | learning |
| Picric acid | + | doubtful | doubtful | + | + | + |
| Dinitroanisole | + | doubtful | + | + | + | + |
| Nitroguanidine | − | doubtful | − | − | − | − |
| PETN | − | doubtful | − | − | − | − |
| Tetryl | + | doubtful | + | + | + | + |
| DTT | + | doubtful | + | + | + | + |
| 1,2-dichloro-4- | + | doubtful | doubtful | − | + | + |
| nitrobenzene | | | | | | |
| 1-chloro-2,4- | + | doubtful | + | − | + | + |
| dinitrobenzene | | | | | | |
| 1-chloro-4- | + | doubtful | doubtful | − | + | + |
| dinitrobenzene | | | | | | |
| 1-methyl-2- | − | doubtful | doubtful | − | doubtful | − |
| nitrobenzene | | | | | | |
| 1-nitro- | + | doubtful | + | + | + | + |
| naphthalene | | | | | | |
| 2,3-DNT | + | doubtful | doubtful | + | + | + |
| 2,4-DNT | + | doubtful | + | + | + | + |
| 2,5-DNT | + | doubtful | + | + | + | + |
| 2,6-DNT | + | doubtful | + | + | + | + |
| 3.4 DNT | + | doubtful | + | + | + | + |
| 3.5 DNT | + | doubtful | + | + | + | + |
| Results | | 0/17 | 12/17 | 14/17 | 16/17 | 17/17 |
| | | | | (3 errors) | | |

Annex 2
AMES mutagenicity test
Different categories of molecules

| | |
|---|---|
| Exp. data | Prediction |

Loads

| | | |
|---|---|---|
| Ammonium dinitramide | + | 67% |
| Picric acid | + | 95% |
| Dinitroanisole | + | 96% |
| NGu | − | 60% |
| PETN | − | 99% |
| Tetryl | + | 94% |
| TNT | + | 94% |
| 1,2-dichloro-4-nitrobenzene | + | 98% |
| 1-chloro-2,4-dinitrobenzene | + | 99% |
| 1-chloro-4-dinitrobenzene | + | 97% |
| 1-methyl-2-nitrobenzene | − | 73% |
| 1-nitro naphthalene | + | 98% |
| 2,3-DNT | + | 90% |
| 2,4-DNT | + | 93% |
| 2,5-DNT | + | 92% |
| 2,6-DNT | + | 90% |
| 3,4 DNT | + | 92% |
| 3,5 DNT | + | 72% |
| 1,3,5 trinitrobenzene | + | 99% |
| 1,3 dinitrobenzene | + | 98% |
| trinitroglycerol | + | 95% |

Plasticizers

| | | |
|---|---|---|
| nitroglycerine | + | 88% |
| TMETN | − | 92% |
| DANPE | + | 82% |
| dibutyl sebacate | − | 76% |
| DEHA | − | 73% |
| DIBP | − | 86% |
| Diallyl phthalate | − | 82% |
| DOTP | − | 83% |
| DOP | − | 82% |
| DEGON | + | 66% |
| EGON | + | 84% |

Oxidizers

| | | |
|---|---|---|
| Acide nitric | + | 68% |
| ammonium nitrate | − | 78% |
| ammonium perchlorate | − | 71% |
| nitrogen tetroxide | + | 57% |
| oxide nitrous | + | 76% |
| HAN | − | 60% |

Liquids

| | | |
|---|---|---|
| Acide perchlorique | − | 64% |
| diethyl ether | − | 71% |
| DMAZ | + | 93% |
| hydrazine | + | 68% |
| methylhydrazine | + | 77% |
| nitrogen tetroxide | + | 61% |
| TMEDA | − | 91% |
| UDMH | + | 73% |

Stabilizers

| | | |
|---|---|---|
| 2-nitro diphenylamine | − | 62% |
| 3-methyl-1,1-diphenylurea | − | 68% |
| calcium carbonate | − | 99% |
| diphenylamine | − | 63% |
| N-methyl-4-nitroaniline | + | 78% |
| triphenylamine | − | 74% |

Pyrotechnics

| | | |
|---|---|---|
| barium chlorate | − | 79% |
| HNS | + | 91% |
| Paris Green | − | 98% |
| sodium nitrate | − | 97% |
| strontium carbonate | − | 100% |
| strontium chloride | − | 93% |
| strontium nitrate | − | 99% |
| strontium sulfate | − | 58% |
| styphnic scid | − | 62% |

Annex 3
Chromosome aberration test
Different categories of molecules

| | |
|---|---|
| Exp. data | Prediction |

loads

| | | |
|---|---|---|
| Picric acid | + | 48% |
| PETN | − | 100% |
| NGu | + | 78% |
| nitrobenzene | + | 100% |
| nitrocellulose | − | 74% |
| 1-chloro-4-nitrobenzene | + | 100% |
| 1-chloro-2,4-dinitrobenzene | + | 92% |
| 1-methyl-4-nitrobenzene | + | 100% |
| 1-methyl-2,4-dinitrobenzene | + | 100% |
| 2,4 DNT | + | 100% |
| 2,6 DNT | + | 95% |
| diisopropyl methylphosphonate | + | 70% |
| Trinitroglycerol | + | 44% |
| 4-nitrotoluene | + | 100% |

plasticizers

| | | |
|---|---|---|
| nitroglycerine | − | 60% |
| DANPE | + | 60% |
| DEHP | + | 100% |
| DEGDN | − | 61% |

liquids

| | | |
|---|---|---|
| MMH | + | 100% |
| UDMH | + | 100% |
| nitrogen tetroxide | + | 84% |
| DMAZ | − | 62% |
| TMEDA | − | 70% |
| hydrazine hydrate | + | 85% |

oxidizers

| | | |
|---|---|---|
| Ammonium nitrate | + | 69% |
| nitrogen tetroxide | + | 83% |

pyrotechnics

| | | |
|---|---|---|
| Paris green | + | 60% |
| sodium nitrate | + | 70% |
| strontium chloride | − | 56% |
| strontium sulfate | − | 53% |

Annex 4
UDS test
Different categories of molecules

| | |
|---|---|
| Exp. data | Prediction |

loads

| | | |
|---|---|---|
| RDX | − | 100% |
| TNT | − | 67% |
| 1-methyl-2-nitrobenzene | + | 100% |
| 1-methyl-4-nitrobenzene | + | 81% |
| 2,4 DNT | + | 100% |
| 2,6 DNT | + | 100% |
| 1,3-DNB | − | D |
| o-cresol | − | 100% |
| 1,4-dioxane | + | 100% |
| 2-nitroluene | − | 84% |

plasticizers

| | | |
|---|---|---|
| DEHP | − | 82% |
| naphthalene | − | 72% |
| DINP | − | 84% |

liquids

| | | |
|---|---|---|
| hydrazine | + | 65% |
| UDMH | − | 63% |
| hydrazine hydrate | + | 60% |

D = doubtful

Annex 5
Carcinogenicity test
Different categories of molecules

| | |
|---|---|
| Exp. data | Prediction |

loads

| | | |
|---|---|---|
| o-cresol | + | 79% |
| 1,4 Dioxane | + | 69% |
| p-cresol | + | 88% |
| 1-methyl-2-nitrobenzene | + | 86% |
| 2,3-DNT | + | 75% |
| 2,4-DNT | + | 78% |
| 2,5-DNT | + | 79% |
| 2,6-DNT | + | 79% |
| 3,4-DNT | + | 81% |
| 3,5-DNT | + | 83% |
| Trinitroglycerol | + | 95% |
| 1-chloro-4-nitrobenzene | + | 92% |
| 1-chloro-2,4-nitrobenzene | + | 89% |
| RDX | + | 57% |
| TNT | − | 70% |
| 1,3,5-trinitrobenzene | − | 88% |

plasticizers

| | | |
|---|---|---|
| butyl benzyl phtalate | + | 81% |
| dibutyl phtalate | − | 90% |
| Diallyl phtalate | + | 57% |
| DEHP | + | 91% |
| DEHA | + | 91% |
| DEGDN | + | 78% |
| naphthalene | + | 93% |

Stabilizers

| | | |
|---|---|---|
| diphenylamine | + | 73% |
| Calcium carbonate | − | 71% |

liquids

| | | |
|---|---|---|
| hydrazine | + | 79% |
| UDMH | + | 100% |
| hydrogen peroxide | + | 93% |
| DMAZ | + | 90% |

Pyrotechnics

| | | |
|---|---|---|
| cadmium oxide | + | 100% |
| hexachloroethane | + | 86% |
| barium chlorate | + | 68% |

Annex 6
Comparison of the results obtained with the prediction
process according to the invention and the ACD process

| | Invention | Invention | |
|---|---|---|---|
| | process | process | ACD method |
| | applied on a | applied on a | applied on the |
| | first test | second test | second test |
| | database | database | database |
| Ames test | 59/61 | 39/45 | 39/45 |
| | | (including 1 | (including 2 |
| | | impossible) | impossible) |
| Chromosome | 26/30 | 22/22 | 16/22 |
| aberration test | | | (including 1 |
| | | | impossible) |
| MLA | 16/18 | 15/15 | 14/15 |
| UDS test | 15/16 | 17/25 | 11/25 |
| | | | (including 2 |
| | | | impossible) |
| DLT assay | 13/16 | 11/12 | Not achievable |
| | | (including 1 | (not developed |
| | | impossible) | by ACD) |
| Carcinogenicity | 30/32 | 34/38 | 19/38 |
| test | | | (including 5 |
| | | | impossible) |
| Reproductive | 18/21 | 23/27 | 1/27 |
| toxicity assay | | | |
| Number of correct | 177/194 | 161/184 | 100/184 |
| answers | | | |
| Percentage (%) of | 91.2 | 87.5 | 54.4 |
| correct answers | | | |

1. Iterative process for selecting a subset of reference molecules to be used to predict at least one property of a target molecular structure, the iterative selection process comprising an initialization step associating with a current molecule a value of a predetermined molecule descriptor associated with the target molecular structure, and during each iteration of the selection process:
a step of evaluating, for each molecule of a database comprising a plurality of molecules each associated with a value of said descriptor, an overall similarity measure between the value of the descriptor associated with said molecule and the value of the descriptor associated with the current molecule;
a step of selecting molecules from the database having an overall similarity measure greater than a predetermined threshold, the selected molecules being added to the reference subset; and
a step of updating the value of the descriptor associated with the current molecule from the values of the descriptors associated with at least some of the molecules belonging to the reference subset.

2. The iterative process according to claim 1 wherein the molecule descriptor comprises N features where N denotes an integer greater than 1, and wherein the evaluation step comprises, for each molecule of the database, a calculation step, for each of the N features of the descriptor, of a local similarity measure between the value of this feature of the descriptor associated with said molecule and the value of this feature of the descriptor associated with the current molecule, the overall similarity measure evaluated for said molecule being obtained from the local similarity measures calculated for this molecule.

3. The iterative process according to claim 2 wherein the calculation step comprises for each feature of the descriptor:
a calculation of a distance between the value of this feature of the descriptor associated with said molecule and the value of this feature of the descriptor associated with the current molecule; and
a conversion of the calculated distance to a real number between 0 and 1 by means of a predetermined conversion function, said number being used as a measure of local similarity for said descriptor feature and said molecule.

4. The iterative process according to claim 3 wherein the calculated distance, noted d, verifies:
$$d ( x , y ) = \{ \begin{matrix}0 if x = y \\ - ∞ if x = 0 and y > 0 \\ + ∞ if x > 0 and y = 0 \\ log ( \frac{x}{y} ) else\end{matrix}$$
where x and y respectively denote the value of the feature of the descriptor associated with said molecule and y denotes the value of the feature of the descriptor associated with the current molecule.

5. The iterative process according to claim 3 wherein the conversion function, noted f, verifies:
$$f ( d ) = exp ( \frac{d}{2 {σ}^{2}} )$$
where d denotes the distance to be converted and σ a predetermined real number.

6. The iterative process according to claim 2 wherein in the evaluation step, the overall similarity measure evaluated for said molecule is the ratio between:
the weighted sum of the N local similarity metrics calculated for the N descriptor features for that molecule, and
twice the sum of the weights applied to the local similarity metrics in said weighted sum less said weighted sum.

7. The iterative process according to claim 2 wherein the values of the N feature of the descriptor reflect the presence or absence of N molecular fragments considered in the definition of a MACCS 166 structural key.

8. The iterative process according to claim 1 wherein, in the update step, the value associated with the current molecule of each descriptor feature is updated with an arithmetic or weighted average of the values of that descriptor feature associated with the molecules of said at least some of the molecules belonging to the reference subset.

9. The iterative process according to claim 1 wherein the molecule descriptor comprises N features where N denotes a number greater than or equal to 1, and wherein, in the update step, the value associated with the current molecule of each feature of the descriptor is updated with the most frequent value of that feature of the descriptor among the values of that feature of the descriptor associated with the molecules of said at least a some of the molecules belonging to the reference subset, or if a plurality of distinct values verify this condition, with the highest value among said plurality of distinct values.

10. The iterative process according to claim 1 wherein in the update step implemented during an iteration of the selection process, said at least some of the molecules belonging to the reference subset include the molecules selected during the selection step of that iteration that were not already part of the reference set before that selection step.

11. The iterative process according to claim 1 wherein in the update step implemented during an iteration of the selection process, said at least some of the molecules belonging to the reference subset include the molecules selected during the selection step of that iteration.

12. The iterative process according to claim 1 wherein in the update step implemented during an iteration of the selection process, said at least some of the molecules belonging to the reference subset comprises all the molecules belonging to the reference subset at the end of the selection step of that iteration.

13. The iterative process according to claim 1 wherein the evaluation, selection and update steps are repeated until a predetermined stopping criterion is verified, said stopping criterion being selected from:
a predetermined number of iterations performed;
a predetermined number of molecules reached in the reference subset;
an absence of molecules selected during the selection step that do not already belong to the reference subset.

14. Process for predicting at least one property of a target molecular substance comprising:
a step of selecting, by means of an iterative selection process according to claim 1, a subset of reference molecules in a database comprising a plurality of molecules each associated with a value of a predetermined molecule descriptor;
a step of predicting at least one property of said target molecular substance from said selected subset of reference molecules.

15. Computer program comprising instructions for performing the steps of the selection process according to claim 1 when said program is executed by a computer.

16. A non-transitory recording medium readable by a computer on which is recorded a computer program comprising instructions for performing the steps of the selection process according to claim 1.

17. Device for selecting a subset of reference molecules for use in predicting at least one property of a target molecular structure, the selection device comprising an initialization module configured to associate with a current molecule a value of a predetermined molecule descriptor, said selection device being further configured to activate, during a plurality of successive iterations:
an evaluation module configured to evaluate, for each molecule of a database comprising a plurality of molecules each associated with a value of the descriptor, a measure of overall similarity between the value of the descriptor associated with said molecule and the value of the descriptor associated with the current molecule;
a selection module configured to select molecules from the database having an overall similarity measure greater than a predetermined threshold, the selected molecules being added by said selection module to the reference subset; and
an update module configured to update the value of the descriptor associated with the current molecule from the values of the descriptors associated with at least some of the molecules belonging to the reference subset.

18. Prediction device, configured to predict at least one property of a target molecular substance comprising:
a selection device in accordance with claim 17, configured to select a subset of reference molecules from a database comprising a plurality of molecules each associated with a value of a predetermined molecule descriptor;
a prediction module, configured to predict at least one property of said target molecular substance from the selected subset of reference molecules.

19. A non-transitory recording medium readable by a computer on which is recorded a computer program comprising instructions for performing the steps of the prediction process according to claim 14.
